(19) United States
(12) Patent Application Publication
(10) Pub. No.: US 2023/0242581 A1
(43) Pub. Date: Aug. 3, 2023

(54) SYNTHESIS OF A GUANYLATE CYCLASE AGONIST BY FRAGMENTS BASED APPROACH

(71) Applicant: ANTHEM BIOSCIENCES PRIVATE LIMITED, Bengaluru, Karnataka (IN)

(72) Inventors: Ganesh SAMBASIVAM, Bengaluru (IN); Atul KASTURCHAND GODHA, Bengaluru (IN); Areti VENKATA BHAGYARAJ, West Godavari dist. (IN); Sathya ANNADURAI, Bengaluru (IN); Avula VEERANJANEYULU, Prakasam (District) (IN); Amol VASANTRAO GADAKH, Bengaluru (IN); Dipak S. JADHAV, Aurangabad District (IN)

(73) Assignee: ANTHEM BIOSCIENCES PRIVATE LIMITED, Bengaluru, Karnataka (IN)

(21) Appl. No.: 18/009,652

(22) PCT Filed: Jun. 15, 2021
(86) PCT No.: PCT/IB2021/055267

§ 371 Date: Dec. 9, 2022

(30) Foreign Application Priority Data
Jun. 17, 2020 (IN) 202041025485

(51) Int. Cl. C07K 5/062; C07K 5/103; C07K 7/06; C07K 7/08

(52) U.S. Cl. CPC C07K 5/0606; C07K 5/1008; C07K 7/06; C07K 7/08

(57) ABSTRACT

The present invention provides a process for the synthesis of Plecanatide, a guanylate cyclase agonist. The process involves convergent synthesis with compounds, i.e., fragment of peptides followed by cyclization. The method provides high yield of Plecanatide with less impurities.

### TECHNICAL FIELD

The present invention is in relation to synthesis of Plecanatide, a Guanylate Cyclase-C(GC-C) agonist. The method of synthesis involves combination of compounds, supposedly fragments of peptides—Plecanatide by convergent approach through solid phase and/or in liquid phase. The method promotes high yield of Plecanatide with high purity.

### BACKGROUND

Plecanatide is a guanylate Cyclase-C(GC-C) agonist, a drug for treatment of chronic idiopathic constipation and irritable bowel syndrome (IBS). The compound increases intestinal transit and fluid through a build-up of 3′,5′-cyclic monophosphate (cGMP). Plecanatide is a 16 amino acid peptide (NDECELCVNVACTGCL), available as a white powder soluble in water. The structure of Plecanatide is represented by formula I.

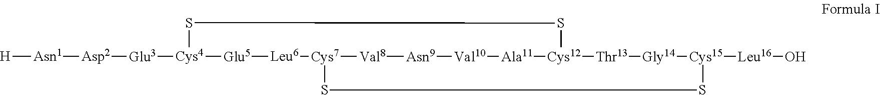

First synthesis of Plecanatide is reported in U.S. Pat. No. 7,041,786, since then many methods are reported for the synthesis of Plecanatide with different approaches such as use of different/multiple resins, coupling reagents, fragments, different protecting group for amino acids; particularly for cysteine, solid/liquid phase and their combination.

U.S. Pat. No. 9,580,471 provides a process for the preparation of Plecanatide, which includes condensation of three fragments. Process is lengthy to prepare fully protected peptide followed by crude Plecanatide. Also, purification protocol after di-cyclization is time consuming to produce Plecanatide.

CN107383170 provides solid phase synthesis of Plecanatide via formation of hexadeca peptide chain onto the resins which is then detached from resin to form linear peptide chain and liquid phase cyclisation is done using air as oxidant. The method is disadvantageous because of low yield and long reaction time.

The methods described in the documents WO2019215753, WO2020034286A1 provide cumbersome and lengthy process with low yield of the Plecanatide.

Considering the importance of Plecanatide as a guanylate Cyclase-C(GC-C) agonist, it is necessary to develop new method/s which can suffice the dire industrial need for synthesis of Plecanatide with acceptable yields and less impurities.

### SUMMARY OF INVENTION

The present invention enables a convergent process for synthesis of Plecanatide. The method comprises solid phase and/or solution phase synthesis of compounds of formula A, A1, B, B1, C, D, D1, E and F detailed below and subsequent condensation of said compounds suitably, in solution and/or on solid phase to form pure linear crude peptide, followed by sequential oxidative cyclization of cysteine amino acid of linear crude peptide to form crude Plecanatide.

More specifically to a method of synthesis of Plecanatide, said method comprising acts of,
a) preparing peptide compound of formula A and deprotecting to obtain peptide compound of formula A1,

Formula A: Fmoc-Cys(Acm)-Leu(OtBu)
Formula A1: Cys(Acm)-Leu(OtBu)

b) coupling peptide compound of formula A1 with peptide compound of formula B or formula D and deprotecting to obtain peptide compound of formula B1 and D1,

Formula B: Fmoc-Ala-Cys(Trt)-Thr(tBu)-Gly-OH
Formula B1: Ala-Cys(Trt)-Thr(tBu)-Gly-Cys(Acm)-
Leu(OtBu)
Formula D: Fmoc-Asn(Trt)-Val-Ala-Cys(Trt)-Thr
(tBu)-Gly-OH
Formula D1: Asn(Trt)-Val-Ala-Cys(Trt)-Thr(tBu)-
Gly-Cys(Acm)-Leu(OtBU)

c) coupling the peptide compound of formula B1 or formula D1 with peptides of formula C or formula E and deprotecting to obtain compound of formula F,

Formula C: Boc-Asn(Trt)-Asp(OtBu)-Glu(OtBu)-Cys
(Trt)-Glu(OtBu)-Leu-Cys(Acm)-Val-Asn(Trt)-Val-OH
Formula E: Boc-Asn(Trt)-Asp(OtBu)-Glu(OtBu)-Cys
(Trt)-Glu(OtBu)-Leu-Cys(Acm)-Val-OH
Formula F: Boc-Asn(Trt)-Asp(otBu)Glu(OtBu)-Cys
(Trt)-Glu(otBu)-Leu-Cys(Acm)-Val-Asn(Trt)-Val-
Ala-Cys(Trt)-Thr(tBu)-Gly-Cys(Acm)-Leu-OtBu

d) oxidising the compound of formula F with charcoal to form disulphide bridge between two Cysteine amino acids at 40 and 12^{th} positions to obtain compound of formula G and lyophilizing;

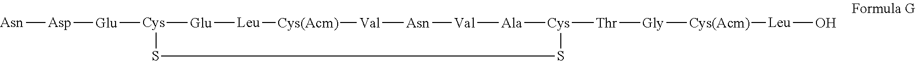

e) treating the lyophilized compound of formula G with Iodine to form second disulphide bridge between two cysteine aminoacids at 7^{th} and 15^{th} positions and quenching with Diaion PAF 308 F resin and purifying to obtain Plecanatide.

The invention is also related to a method of preparation of Plecanatide comprising acts of
a) coupling peptide compounds selected from a group containing

(i) Formula A1:
Cys(Acm)-Leu(OtBu),
(ii) Formula B:
Fmoc-Ala-Cys(Trt)-Thr(tBu)-Gly-OH,
(iii) Formula B1:
Ala-Cys(Trt)-Thr(tBu)-Gly-Cys(Acm)-Leu(OtBu),
(iv) Formula D:
Fmoc-Asn(Trt)-Val-Ala-Cys(Trt)-Thr(tBu)-Gly-OH,
(v) Formula D1:
Asn(Trt)-Val-Ala-Cys(Trt)-Thr(tBu)-Gly-Cys(Acm)-
Leu(OtBU),
(vi) Formula C:
Boc-Asn(Trt)-Asp(OtBu)-Glu(OtBu)-Cys(Trt)-Glu
(OtBu)-Leu-Cys(Acm)-Val-Asn(Trt)-Val-OH,and
(vii) Formula E:
Boc-Asn(Trt)-Asp(OtBu)-Glu(OtBu)-Cys(Trt)-Glu
(OtBu)-Leu-Cys(Acm)-Val-OH;

to form compound of formula F,
b) oxidising the compound of formula F with charcoal to form disulphide bridge between two Cysteine amino acids at 4^{th} and 12^{th} positions to obtain compound of formula G and lyophilizing, and

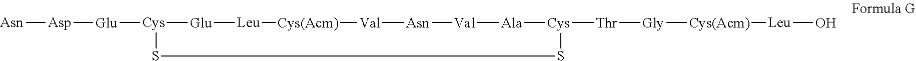

c) treating the lyophilized compound of formula G with Iodine to form second disulphide bridge between two cysteine aminoacids at 7^{th} and 15^{th} positions and quenching with Diaion PAF 308 F resin and purifying to obtain Plecanatide.

The invention also provides peptide compound of formula B and its preparation in a facile cost effective manner for adoption in the preparation of plecanatide.

Formula B:
Fmoc-Ala-Cys(Trt)-Thr(tBu)-Gly-OH.

The invention also provides peptide compound of formula C and its facile preparation in a facile cost effective manner for adoption in the preparation of Plecanatide.

Formula C:
Boc-Asn(Trt)-Asp(OtBu)-Glu(OtBu)-Cys(Trt)-Glu
(OtBu)-Leu-Cys(Acm)-Val-Asn(Trt)-Val-OH

The invention also provides a peptide compound of formula D and its facile method of preparation for adoption in the preparation of plecanatide.

Formula D:
Fmoc-Asn(Trt)-Val-Ala-Cys(Trt)-Thr(tBu)-Gly-OH

The invention also provides a peptide compound of formula E and its facile method of preparation for adoption in the preparation of plecanatide.

Formula E:
Boc-Asn(Trt)-Asp(OtBu)-Glu(OtBu)-Cys(Trt)-Glu
(OtBu)-Leu-Cys(Acm)-Val-OH

The selected group of compounds in solution or on solid phase approach of present invention enables convenient synthesis of Plecanatide with good yield and high purity (>98%) on an industrial scale with efficient turnaround time.

### DESCRIPTION OF INVENTION

The present invention may be understood by the reference to the description and examples; which are intended to exemplify non limiting embodiment of the present invention.

The invention is in relation to method of preparation of Plecanatide by fragment synthesis approach. The convergent synthesis which includes solid phase as well as liquid phase of synthesis is followed by purification as provided below.

An embodiment of the method of synthesis comprises combination of compounds, which are peptide fragments of Plecanatide with necessary protection groups and cyclization to obtain the target compound Plecanatide. The compounds are prepared by solid phase and liquid phase methods and suitably combined to minimize the impurities and increase the yield. The peptide fragment compounds for the synthesis of Plecanatide are selected from a group comprising compounds of—

Formula A:
Fmoc-Cys(Acm)-Leu-OtBu;
Formula B:
Fmoc-Ala-Cys(Trt)-Thr(tBu)-Gly-OH;
Formula C:
Boc-Asn(Trt)-Asp(OtBu)-Glu(OtBu)-Cys(Trt)-Glu
(OtBu)-Leu-Cys(Acm)-Val-Asn(Trt)-Val-OH;
Formula D:
Fmoc-Asn(Trt)-Val-Ala-Cys(Trt)-Thr(tBu)-Gly-OH;
Formula E:
Boc-Asn(Trt)-Asp(OtBu)-Glu(OtBu)-Cys(Trt)-Glu
(OtBu)-Leu-Cys(Acm)-Val-OH;
Formula A1:
Cys(Acm)-Leu(OtBu)
Formula B1:
Ala-Cys(Trt)-Thr(tBu)-Gly-Cys(Acm)-Leu(OtBu)
Formula D1:
Asn(Trt)-Val-Ala-Cys(Trt)-Thr(tBu)-Gly-Cys(Acm)-
Leu(OtBU)

The above mentioned compounds are prepared by liquid phase/solid phase peptide synthesizer easily on industrial scale without any difficulty with good purity and in high yield. The fragments in liquid phase to provide desired protected linear peptide and subsequently desired final Plecanatide with good yield and purity.

In another embodiment of the invention the compound of formula A: Fmoc-Cys(Acm)-Leu-OtBu is prepared by liquid phase. The other compound of formula B to E: Fmoc-Ala-Cys(Trt)-Thr(tBu)-Gly-OH; Boc-Asn(Trt)-Asp(OtBu)-Glu(OtBu)-Cys(Trt)-Glu(OtBu)-Leu-Cys(Acm)-Val-Asn(Trt)-Val-OH; Fmoc-Asn(Trt)-Val-Ala-Cys(Trt)-Thr(tBu)-Gly-OH; Boc-Asn(Trt)-Asp(OtBu)-Glu(OtBu)-Cys(Trt)-Glu(OtBu)-Leu-Cys(Acm)-Val-OH; are prepared by solid phase synthesis.

In an embodiment two different approaches are designed to prepare the target compound adopting suitable peptide fragments. The above said compounds B, B1, C, D, D1 and E in with very minor or no racemization in liquid phase provide desired protected linear peptide and subsequently Plecanatide with good yield and purity.

In an embodiment, the method of synthesis of Plecanatide, comprises steps of,
a) preparing peptide compound of formula A and deprotecting to obtain peptide compound of formula A1,

Formula A:
Fmoc-Cys(Acm)-Leu(OtBu)
Formula A1:
Cys(Acm)-Leu(OtBu)

b) coupling peptide compound of formula A1 with peptide compound of formula B or formula D and deprotecting to obtain peptide compound of formula B1 and D1,

Formula B:
Fmoc-Ala-Cys(Trt)-Thr(tBu)-Gly-OH
Formula B1:
Ala-Cys(Trt)-Thr(tBu)-Gly-Cys(Acm)-Leu(OtBu)
Formula D:
Fmoc-Asn(Trt)-Val-Ala-Cys(Trt)-Thr(tBu)-Gly-OH
Formula D1:
Asn(Trt)-Val-Ala-Cys(Trt)-Thr(tBu)-Gly-Cys(Acm)-
Leu(OtBU)

c) coupling the peptide compound of formula B1 or formula D1 with peptides of formula C or formula E and deprotecting to obtain compound of formula F,

Formula C:
Boc-Asn(Trt)-Asp(OtBu)-Glu(OtBu)-Cys(Trt)-Glu
(OtBu)-Leu-Cys(Acm)-Val-Asn(Trt)-Val-OH
Formula E:
Boc-Asn(Trt)-Asp(OtBu)-Glu(OtBu)-Cys(Trt)-Glu
(OtBu)-Leu-Cys(Acm)-Val-OH
Formula F:
Boc-Asn(Trt)-Asp(otBu)Glu(OtBu)-Cys(Trt)-Glu
(otBu)-Leu-Cys(Acm)-Val-Asn(Trt)-Val-Ala-Cys
(Trt)-Thr(tBu)-Gly-Cys(Acm)-Leu-OtBu

d) oxidising the compound of formula F with charcoal to form disulphide bridge between two Cysteine amino acids at 40 and 12^{th} positions to obtain compound of formula G and lyophilizing;

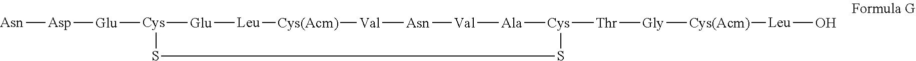

e) treating the lyophilized compound of formula G with Iodine to form second disulphide bridge between two cysteine aminoacids at 7^{th} and 15^{th} positions and quenching with Diaion PAF 308 F resin and purifying to obtain Plecanatide.

In still another embodiment, the method of preparation of Plecanatide comprises steps of
a) coupling peptide compounds selected from a group containing

(i) Formula A1:
Cys(Acm)-Leu(OtBu),
(ii) Formula B:
Fmoc-Ala-Cys(Trt)-Thr(tBu)-Gly-OH,
(iii) Formula B1:
Ala-Cys(Trt)-Thr(tBu)-Gly-Cys(Acm)-Leu(OtBu),
(iv) Formula D:
Fmoc-Asn(Trt)-Val-Ala-Cys(Trt)-Thr(tBu)-Gly-OH,
(v) Formula D1:
Asn(Trt)-Val-Ala-Cys(Trt)-Thr(tBu)-Gly-Cys(Acm)-
Leu(OtBU),
(vi) Formula C:
Boc-Asn(Trt)-Asp(OtBu)-Glu(OtBu)-Cys(Trt)-Glu
(OtBu)-Leu-Cys(Acm)-Val-Asn(Trt)-Val-OH,
and
(vii) Formula E:
Boc-Asn(Trt)-Asp(OtBu)-Glu(OtBu)-Cys(Trt)-Glu
(OtBu)-Leu-Cys(Acm)-Val-OH;

to form compound of formula F: Boc-Asn(Trt)-Asp(otBu)Glu(OtBu)-Cys(Trt)-Glu(otBu)-Leu-Cys(Acm)-Val-Asn(Trt)-Val-Ala-Cys(Trt)-Thr(tBu)-Gly-Cys(Acm)-Leu-OtBu,
b) oxidising the compound of formula F with charcoal to form disulphide bridge between two Cysteine amino acids at 4^{th} and 12^{th} positions to obtain compound of formula G and lyophilizing, and

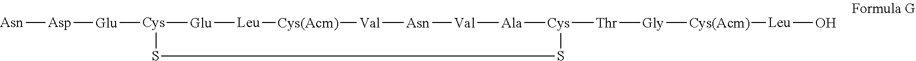

c) treating the lyophilized compound of formula G with Iodine to form second disulphide bridge between two cysteine amino acids at 7^{th} and 15^{th} positions and quenching with Diaion PAF 308 F resin and purifying to obtain Plecanatide.

In an embodiment, three compounds—Formula-A: Fmoc-Cys(Acm)-Leu-OtBu synthesized in liquid phase, Formula B: Fmoc-Ala-Cys(Trt)-Thr(tBu)-Gly-OH; Formula C: Boc-Asn(Trt)-Asp(OtBu)-Glu(OtBu)-Cys(Trt)-Glu(OtBu)-Leu-Cys (Acm)-Val-Asn(Trt)-Val-OH synthesized by solid phase independently are condensed in sequential manner and cyclized as described in flow chart-1.

In still another embodiment, three compounds-Formula A: Fmoc-Cys(Acm)-Leu-OtBu synthesized in liquid phase, Formula D: Fmoc-Asn(Trt)-Val-Ala-Cys(Trt)-Thr(tBu)-Gly-OH; Formula E: Boc-Asn(Trt)-Asp(OtBu)-Glu(OtBu)-Cys(Trt)-Glu(OtBu)-Leu-Cys(Acm)-Val-OH synthesized by solid phase independently are condensed in sequential manner and cyclized as described in flow chart −2.

In an embodiment, all amino acids adopted for the synthesis are Fmoc-AA-OH or Boc-AA-OH with orthogonal protecting groups.

In an embodiment, solid-phase synthesis is carried out with resins selected from a group comprising Tentagel, Wang resin and 2-CTC resin.

Loading of resin is preferably in the range of 0.3-1.6 mmol/g. The resin is swelled with a solvent before use. Cleavage of resin is carried out by using mixed solution of TFA: DCM (1-2%:98%) to react with the resin for 3-4 times, each time for 10-20 minutes.

The solvents for the reactions are selected from a group comprising Dimethyl formamide, N-Methyl-2-pyrrolidone, dichloromethane, N, N-dimethylacetamide (DMA), Acetonitrile, Ethylene dichloride (EDC) tetrahydrofuran (THF), 2-Methyl tetrahydrofuran and N-butylpyrrolidinone.

The coupling reaction of each amino acid is varied ranging from about 2 to about 6 hours, preferably 2 to 4 hours at temperature ranging from about 20° C. to about 35° C.; or at suitable elevated temperature.

Fmoc group is removed using a reagent selected from the group comprising, 20% Piperidine in DMF, 20% 4-Methyl Piperidine in DMF, DBU in DMF and Tert.butyl amine, Diethyl amine in DMF for 5 minutes followed by 15 minutes. Coupling agents are selected from a group comprising DIPC (Di-isopropyl carbodimide), 2-(1H-Benzotriazole-1-yl)-1,1,3,3-tetramethyluronium tetrafluoroborate (TBTU), 2-(1H-benzotriazol-1-yl)-1,1,3,3-tetramethyluronium Hexafluorophosphate (HBTU), 1-[Bis(dimethylamino)methylene]-1H-1,2,3-triazolo[4,5-b] pyridinium 3-oxide hexafluorophosphate (HATU), Hydroxybenzotriazole (HOBt), 6-chloro HOBt, Cyano[morpholino(dimethyliminio)methoxyimino]acetic acid ethyl ester (COMU), 1-Hydroxy-7-azabenzotriazole (HOAt) and the like.

In another embodiment, global deprotection are carried out with Trifluoroacetic acid (TFA): Triisopropyl silane (TIPS): Phenol: H₂O mixture mixed in a proportion 88%, 5.0%, 5.0%, and 2.0% respectively or a mixture of TFA: TIPS: Phenol: Dithiothreitol: Thioanisole: H₂O mixed in a proportion of 82.5%, 2.5%, 2.5%, 2.5%, 5%, 5% respectively.

First disulfide cyclization is carried out with charcoal in ACN: H₂O for about 1 hour, later it is purified by reverse-phase high-pressure liquid chromatography to obtain pure monocyclised peptide.

Second disulfide cyclization reaction is carried with I₂ in 5% acetic acid for 1 hour and the excess Iodine is quenched with Diaion PAF 308 F resin. The cyclization is followed by purification carried out by reverse-phase high-pressure liquid chromatography which includes octadecylsilane as a stationary phase and 0.1% trifluoro acetic acid aqueous solution/acetonitrile as a mobile phase and fractions are pooled for lyophilization to obtain pure Plecanatide.

To exemplify the synthesis, the method and details adopted involving compounds of formula A, B and C are given below.

The general method adopted for coupling of peptide fragments in liquid phase is given below in schematic diagrams. All amino acids are commercially purchased from Chem-Impex, CS Bio, GL Bio Advance ChemTech, and the like. The synthesis of peptides is carried out by manual or in CS Bio peptide synthesizer (CS536XT).

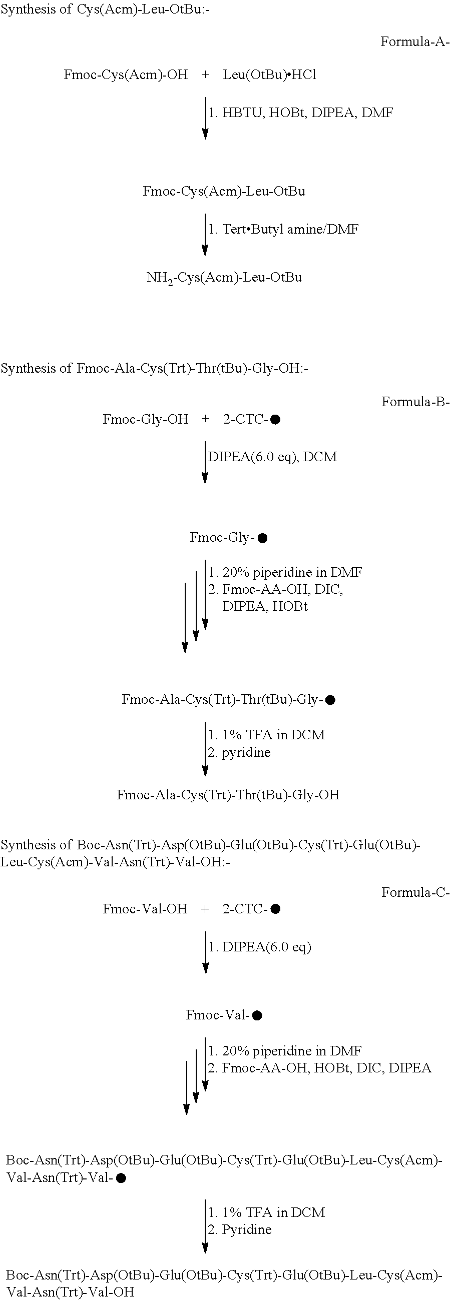

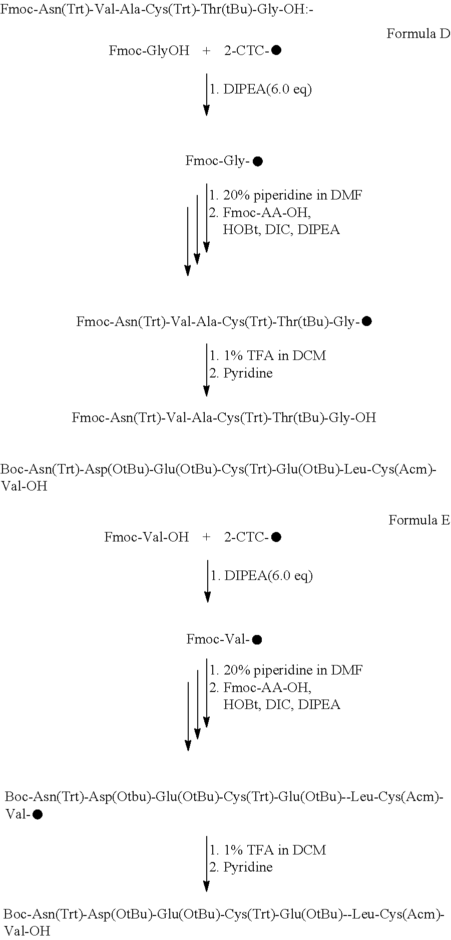

In an embodiment, the three fragment approach adopting compounds of present invention i.e., of formula A, A1, B, B1, C, D, D1, E for synthesis of Plecanatide as in flow chart-1 and flow chart 2, substantially reduce turnaround time to expedite the synthesis on industrial scale. The compounds—B, C, D and E) are synthesized with fully automated solid phase peptide synthesizer. After synthesis, the compounds are coupled in solution phase without any special equipment.

In another embodiment for global deprotection step, eco-friendly reagents such as Dithiothreitol (DTT) is adopted. DTT is also advantageous in handling on an industrial scale due to its solid nature.

In another embodiment, the first disulphide formation involves granular charcoal to produce more stable and quality intermediate of formula G.

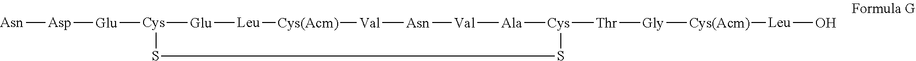

The purification at this stage, results in first disulphide intermediate with over 97% purity and could avoid stringent purification at later stage.

In still another embodiment, the excess Iodine is quenched with Diaion PAF 308 F resin, which is easily removable after filtration during second disulphide bridge formation. The overall yield of about 60-70% with high purity about 98%-98.5% is achieved by the method.

### EXPERIMENTAL

### A. Fragment Compounds Synthesis—

### I. Synthesis of Fmoc-Ala-Cys (Trt)-Thr(tBu)-Gly-OH

### Example 1

Said compound is synthesized using 2-CTC resin by Fmoc solid phase peptide strategy. 2-CTC resin (100 g, loading capacity 0.62 mmol/g, 100-200 mesh) is charged in a 2000 mL clean dry peptide synthesizer vessel. Resin is swelled in DCM (10V) for 30 min, 2 times each.

1^{st} amino acid coupling: To a pre-swelled resin, previously prepared solution of first amino acid Fmoc-Gly-OH (55.2 g, 3.0 eq) in DCM (10 Vol.) along with DIPEA (64.7 mL, 6.0 eq) is added and agitated for 6 hr at room temperature. After 6 hr, solvent is drained, resin is washed with DCM (2×5 Vol.). Mixture of DCM: MeOH: DIPEA (85:10:5, 10 Vol.) is added to resin and agitated for 15 min. Solvent is drained and resin is washed with DCM (2×5 Vol.) followed by DMF (2×5 Vol.).

Fmoc Deprotection: Deprotection of Fmoc group is performed by adding 20% Piperidine in DMF (2×10 Vol.) for 10 min followed by 15 min. Solvent is drained, resin is washed with DMF (2×5 Vol.) followed by DCM (2×5 Vol.) and DMF (2×5 Vol.)

2^{nd} amino acid coupling: The pre-activated Fmoc-Thr(tBu)-OH (49.2 g, 2.0 eq) with HOBt (16.7 g, 2.0 eq) and DIC (19.2 mL, 2.0 eq) in DMF (10 V) is added to above resin. Coupling is continued for 3 hr. Solvent is drained and peptide resin is washed with DMF (2×5 Vol.), DCM (2×5 Vol.) and DMF (2×5 Vol.) respectively. Sequential coupling of next two amino acids Fmoc-Cys(trt)-OH (72.6 g, 2.0 eq),

Fmoc-Ala-OH (38.6 g, 2.0 eq) are performed similar to the above procedure. Fmoc deprotection is carried out similar to the above procedure.

After each coupling and Fmoc deprotection, peptide resin is washed with DMF (2×5 Vol.), DCM (2×5 Vol.) and DMF (2×5 Vol.) respectively.

Each coupling and deprotection is monitored by Bromophenol blue test.

After completion of synthesis, peptide resin is washed with Methanol (2×5 Vol.), MTBE (2×5 Vol.) and DCM (2×5 Vol.) respectively.

Resin cleavage: Peptide is cleaved from the resin using 1% TFA/DCM (10 Vol. X 3 times), 5 minutes for each time. Cleavage fraction is filtered and neutralised with pyridine each time (1:1 Vol. to TFA). Combined cleavage fractions are concentrated under vacuum at 30-38° C. The resulting solution is co-evaporated with ethanol (2×5 Vol.) up to minimum volume and slurry is added in to purified water, stirred for 18 h. Product is isolated by filtration, washed with purified water and suck dried for 24 h. The solid is dried in VTD under reduced pressure at 40-45° C. to afford white solid. Yield: 43.20 g, 80.1%

II. Synthesis of Boc-Asn(Trt)-Asp(OtBu)-Glu(OtBu)-Cys(Trt)-Glu(OtBu)-Leu-Cys (Acm)-Val-Asn(Trt)-Val-OH

### Example 2

The compound is synthesized using 2-CTC resin by Fmoc solid phase peptide strategy.

2-CTC resin (100 g, loading capacity-0.62 mmol/g) is charged in a 3000 mL clean dry peptide synthesizer vessel. Resin is swelled with DCM (10V), 30 min, 2 times each.

1^{st} amino acid coupling: First amino acid Fmoc-Val-OH (63.12 g, 3.0 eq) with DIPEA (64.7 mL, 6.0 eq) in DCM (10 Vol.) is added to pre-swelled resin; agitated for 6 h. Drained the solvent, washed the resin with DCM (2×5 Vol.) Capping solution of DCM: MeOH: DIPEA (85%:10%:5%, 10 Vol.) is charged to above resin, agitated for 15 min for capping the resin. Drained the solvent and resin is washed with DCM (2×5 Vol.) and DMF (2×5 Vol.) respectively.

Fmoc deprotection: Deprotection of Fmoc group is performed by adding 20% piperidine in DMF (2×10 Vol.) for 10 min followed by 15 min respectively. Drained the solvent, washed resin with DMF (2×5 Vol.), DCM (2×5 Vol.) and DMF (2×5 Vol.) respectively.

2^{nd} amino acid coupling: The pre-activated Fmoc-Asn(trt)-OH (73.9 g, 2.0 eq) with HOBt (16.7 g, 2.0 eq) and DIC (19.2 mL, 2.0 eq) in DMF (10V) is added to loaded resin, agitated for 3 h. Drained solvent and peptide resin is washed with DMF (2×5 Vol.), DCM (2×5 Vol.), DMF (2×5 Vol.) respectively.

Sequential coupling of next eight amino acids Fmoc-Val-OH (42.08 g, 2.0 eq), Fmoc-Cys(Acm)-OH (51.3 g, 2.0 eq), Fmoc-Leu-OH (43.8 g, 2.0 eq), Fmoc-Glu(OtBu)-OH (54.9 g, 2.0 eq), Fmoc-Cys(trt)-OH (72.62 g, 2.0 eq), Fmoc-Glu(OtBu)-OH (54.9 g, 2.0 eq), Fmoc-Asp(OtBu)-OH (51.0 g, 2.0 eq) and Boc-Asn (Trt)-OH (58.8 g, 2.0 eq) are performed similar to the above procedure.

After each coupling and deprotection the peptide resin is washed DMF (2×5 Vol.), DCM (2×5 Vol.) and DMF (2×5 Vol.) respectively.

Each coupling and deprotection is monitored by Bromophenol blue test.

After completion of synthesis, peptide resin is washed with Methanol (2×5 Vol.), MTBE (2×5 Vol.) and DCM (2×5 Vol.) respectively.

Resin cleavage: Peptide is cleaved from the resin using 1% TFA/DCM (3×10 Vol.), 5 minutes for each time. Cleavage fractions are collected neutralized with pyridine (1:1 Volume ratio to TFA in each cleavage). Combined cleavage fractions are concentrated under vacuo at 30-38° C. Resulting solution is co-evaporated with ethanol (2×5 Vol.) up to minimum volume and slurry is added to purified water, stirred for 18 h, solid is filtered, washed with purified water and suck dried for 24 h and then dried in VTD under reduced pressure at 45-50° C. to afford white solid. Yield: 116.6 g, 85.4%

### B. Coupling of Compounds

III. Synthesis of Fmoc-Cys(Acm)-Leu(OtBu):

### Example 3

To a stirred solution of Fmoc-Cys(Acm)-OH (200 g, 1 eq) in DMF (10 Vol), Leu(OtBu). HCl (107 g, 1 eq), HOBt (65 g, 1 eq) and HBTU (182 g, 1 eq) are added by maintaining temperature 0-10° C. DIPEA (294 ml, 3.5 eq) is added slowly by maintaining pH6-7 at same temperature. Reaction mixture is stirred for 3 hrs at room temperature. The reaction mixture is diluted with ethyl acetate (2 Lit) and washed with 5% H₃PO₄ (700 ml), saturated NaHCO₃ (2×500 ml), purified water (2×1 1) and saturated NaCl (2×11). Organic layer is dried over anhydrous Na₂SO₄, filtered and concentrated under reduced pressure to give a white crystalline solid which is recrystallized from ethyl acetate/hexane to obtain pure dipeptide (250 g, 89% yield) with HPLC purity >99%.

### Example 4

To a stirred solution of Fmoc-Cys(Acm)-OH (50 g, 1 eq) in DMF (500 mL, 10 Vol.) is added Leu(OtBu). HCl (26.9 g, 1 eq) followed by addition of HOBt (16.3 g, 1.0 eq), HBTU (45.7 g, 1.0 eq) is carried by maintaining temperature 0° C.-10° C. DIPEA (73.5 g, 3.5 eq) is added in a drop wise manner by maintaining pH 6-7 at same temperature. Reaction mixture is stirred at 0° C.-10° C. for 30 min. Reaction mixture is stirred for 3 hrs at 25-30° C. After completion of the reaction, reaction mixture is diluted with ethyl acetate (15 Vol.) and washed with 5% H₃PO₄ (8 Vol.), saturated NaHCO₃ (8 Vol.) solution and saturated NaCl solution. Organic layer is dried over anhydrous Na₂SO₄, filtered and concentrated under reduced pressure to dryness. The product is recrystallized from ethyl acetate/hexane to afford Fmoc-Cys(Acm)-Leu(OtBu) as a white solid. Yield: 54 g, 76.7%

IV. Deprotection to Form Cys(Acm)-Leu(OtBu)

### Example 5

Fmoc-Cys(Acm)-Leu(OtBu) (25 g, 1 eq), is dissolved in Tert. Butyl amine/DMF solution (5 Vol) and stirred for 3 hrs. Reaction mixture is directly concentrated to remove volatile solvent under reduced pressure. To the residue, hexane (250 ml) is added, stirred and supernatant liquid is decanted. The residue is dissolved in ethyl acetate (500 ml), washed with NaH₂PO₄/Na₂HPO₄ solution (200 ml) and saturated NaCl solution (200 ml). The organic layer is dried over anhydrous Na₂SO₄, filtered and concentrated under reduced pressure to obtain oily product (10 g, 64% yield) with LCMS purity >95%.

### Example 6

To an ice cooled stirred solution of Fmoc-Cys(Acm)-Leu(OtBu) (50.0 g, 1.0 eq) in DMF (10 Vol.) is added Tertiary butyl amine (9.38 g, 1.5 eq) and stirred for 15 min at 5-10° C. n-heptane (5 Vol.) is charged into the reaction mass, stirred for 2 hr at 25-30° C. After completion of reaction, n-heptane layer is separated and DMF layer is cooled to 0-10° C., pH is adjusted to 3-4 by using 3% KHSO₄ solution (1.1 L) then washed with 6% Ethyl acetate in Heptane (3×5 Vol.). Aqueous layer pH is adjusted to 6-7 by using 2.5% NaHCO₃ solution, again washed with 6% Ethyl acetate in Heptane (3×5 Vol.). The product is extracted using DCM (10 Vol., 5 Vol.×2). Organic layer is dried over anhydrous Na₂SO₄, filtered and concentrated under reduced vacuum to afford Cys (Acm)-Leu (OtBu) as yellow oily mass. Yield: 24.1 g and 77.49%

V. Coupling of Fmoc-Ala-Cys(Trt)-Thr(tBu)-Gly-OH with Cys(Acm)-Leu(OtBu)

### Example 7

To a stirred solution of Fmoc-Ala-Cys(Trt)-Thr(tBu)-Gly-OH (5 g, 1 eq) in DMF (15 vol), Cys(Acm)-Leu(OtBu) (2.07 g, 1 eq), Cl-HOBt (1.16 g, 1.2 eq) and HBTU (2.61 g, 1.2 eq) are added by maintaining temperature −5 to 10° C. DIPEA (2.9 ml, 3 eq) is added slowly by maintaining pH 6-7. Stirring is continued for 30 minutes at −5 to 10° C. Resulting reaction mixture is stirred for about 3 to 4 hrs at 25-30° C. After completion of the reaction, reaction mixture is poured into precooled 0.5N HCl (10 vol), stirred for a period of 15-30 minutes. The solid is filtered under vacuum, washed with 0.5N HCl (2×50 vol), purified water (2×50 vol), Sat. NaHCO₃ (2×50 vol), purified water (2×50 vol) and MTBE (2×50 vol). The solid is dried under high vacuum to obtain peptide (4.3 g, 62%) with LCMS purity >99%.

### Example 8

To a stirred solution of Fmoc-Ala-Cys(Trt)-Thr(tBu)-Gly-OH (70.0 gm, 1.0 eq) in DMF (10 Vol.), is added Cys(Acm)-Leu(OtBu) (34.88 g, 1.2 eq) and HOAt (13.12 g, 1.2 eq) by maintaining temperature 5 to 10° C. EDC HCl (23.12 g, 1.5 eq) is added in a lot wise manner at same temperature. Resulting reaction mixture is stirred for about 3 to 4 hrs at 25-30° C. After completion of the reaction, reaction mixture is poured into pre-cooled 2.5% NaHCO₃ solution (2.8 L, 40 Vol.), stirred for a period of 15-30 minutes. Solid is filtered under vacuum, washed with purified water (350 mL). The obtained solid is leached with 1% KHSO₄ solution (700 mL, 10 Vol.) and ACN: Water (5:10, 1.05 L, 15 V), filtered and dried under high vacuum to obtain off white solid.

Yield: 97.59 g, 93.24%

VI. Deprotection to Form Ala-Cys(Trt)-Thr(tBu)-Gly-Cys(Acm)-Leu(OtBu):

### Example 9

To a stirred solution of Fmoc-Ala-Cys(Trt)-Thr(tBu)-Gly-Cys(Acm)-Leu(OtBu) (4.3 g, 1 eq) in DMF (30 vol), Piperidine (13 ml) is added by maintaining temperature 5 to 10° C. Stirring is continued for 3-4 hr and reaction is monitored by TLC. After completion, reaction mixture is concentrated to remove the solvent under reduced pressure. To the residue hexane (43 ml, 10 vol) is added, stirred and the supernatant liquid is decanted. The white precipitate is collected by filtration to obtain product (2.9 g, 82% yield) with LCMS purity >90%.

### Example 10

To an ice cooled stirred solution of Fmoc-Ala-Cys(Trt)-Thr(tBu)-Gly-Cys(Acm)-Leu(OtBu) (50.0 g, 1.0 eq) in DMF (500 mL, 10 Vol.), Tertiary butyl amine (6.35 mL, 1.5 eq) is added, stirred for 15 min at 5-10° C. n-heptane (250 mL, 5 Vol.) is charged into reaction mixture, stirred for 4 hr at 25-30° C. After completion of reaction, n-heptane layer is separated and DMF layer is cooled to 0-10° C., pH is adjusted to 3-4 by using 20% KHSO₄ solution (150 mL) and filtered. Filtrate is washed with 30% MTBE/Heptane (3×5 Vol.) and Ethyl acetate/Heptane (3×5 Vol.). Filtrate is cooled to 0-10° C., pH is adjusted to 6-7 by using 2.5% NaHCO₃ solution (200 mL). Desired product is extracted using DCM (lx 10 Vol., 2×5 Vol.). Combined organic layer is dried over anhydrous Na₂SO₄, filtered and concentrated under reduced vacuum, solid isolated in MTBE (10 Vol.) to afford off white solid. Yield: 40.87 g, 85.88%

VI. Coupling of Boc-Asn(Trt)-Asp(OtBu)-Glu(OtBu)-Cys(Trt)-Glu(OtBu)-Leu-Cys(Acm)-Val-Asn(Trt)-Val-OH with Ala-Cys(Trt)-Thr(tBu)-Gly-Cys(Acm) Leu(OtBu):

### Example 11

To a stirred solution of Boc-Asn(Trt)-Asp(OtBu)-Glu(OtBu)-Cys(Trt)-Glu(OtBu)-Leu-Cys(Acm)-Val-Asn(Trt)-Val-OH (3 g, 1 eq) in DMF (25 vol), Ala-Cys(Trt)-Thr(tBu)-Gly-Cys(Acm)-Leu(OtBu)(1 eq), Cl-HOBt (1.2 eq) and HBTU (1.2 eq) are added by maintaining temperature −5 to 10° C. DIPEA is added slowly by maintaining pH 6-7. Stirring is continued for 30 minutes at −5 to 10° C. The reaction mixture is stirred for 3-4 hrs at 25-30° C. After completion of the reaction, reaction mixture is poured into precooled 0.5N HCl solution, stirred for a period of 15-30 minutes. Solid is filtered under vacuum, washed with 0.5N HCl (2×25 vol), purified water (2×25 vol), Sat. NaHCO₃ (2×25 vol), purified water (2×25 vol) and MTBE (2×25 vol). The solid is dried under high vacuum to obtain fully protected peptide (˜78% yield) with LCMS purity >95%.

### Example 12

To a stirred solution of Boc-Asn(Trt)-Asp(OtBu)-Glu(OtBu)-Cys(Trt)-Glu(OtBu)-Leu-Cys(Acm)-Val-Asn(Trt)-Val-OH (97.71 g, 1.1 eq) in DMF (1.0 L, 25 Vol.) is added Ala-Cys(Trt)-Thr(tBu)-Gly-Cys(Acm)-Leu-OtBu (40 g, 1.0 eq) followed by addition of Cl-HOBt (17.09 g, 2.5 eq), HBTU (38.22 g, 2.5 eq) by maintaining temperature −5 to 10° C. and DIPEA (21.5 mL, 3.0 eq) is added slowly by maintaining pH 6-7 at same temperature. Reaction mixture is stirred for 30 minutes at −5 to 10° C. for 3-4 hrs at 25-30° C. After completion of the reaction, reaction mixture is poured into pre-cooled methanol (5.0 L), stirred for a period of 2-3 h. Solid is filtered over Buchner funnel, washed with methanol (400 mL) and dried. Solid is slurred with hexane (20 V) for 1 h, filtered and dried under high vacuum to obtain fully protected peptide as white solid.

Yield: 113 g, 88.18%.

VI. Synthesis of Fmoc-Asn(Trt)-Val-Ala-Cys(Trt)-Thr(tBu)-Gly-OH—

### Example 13

2-CTC resin (100 g, loading capacity-1.1 mmol/g) is charged in a 3000 mL clean dry peptide synthesizer vessel. Resin is swelled with DCM (10V), 30 min, 2 times each.

1^{st} amino acid coupling: First amino acid Fmoc-Gly-OH (98.13 g, 3.0 eq) with DIPEA (85.3 mL, 6.0 eq) in DCM (10 Vol.) is added to pre-swelled resin; agitated for 6 h. Drained the solvent, washed the resin with DCM (2×5 Vol.) Capping solution of DCM: MeOH: DIPEA (85%:10%:5%, 10 Vol.) is charged to above resin, agitated for 15 min for capping the resin. Drained the solvent and resin is washed with DCM (2×5 Vol.) and DMF (2×5 Vol.) respectively.

Fmoc deprotection: Deprotection of Fmoc group is performed by adding 20% piperidine in DMF (2×10 Vol.) for 10 min followed by 15 min respectively. Drained the solvent, washed resin with DMF (2×5 Vol.), DCM (2×5 Vol.) and DMF (2×5 Vol.) respectively.

2^{nd} amino acid coupling: The pre-activated Fmoc-Thr(tBu)-OH (87.44 g, 2.0 eq) with HOBt (29.73 g, 2.0 eq) and DIC (27.76 mL, 2.0 eq) in DMF (10V) is added to loaded resin, agitated for 3 h. Drained solvent and peptide resin is washed with DMF (2×5 Vol.), DCM (2×5 Vol.), DMF (2×5 Vol.) respectively.

Sequential coupling of next four amino acids Fmoc-Cys(trt)-OH (128.85 g, 2.0 eq), Fmoc-Ala-OH (68.49 g, 2.0 eq), Fmoc-Val-OH (74.66 g, 2.0 eq), Fmoc-Asn (Trt)-OH (131.26 g, 2.0 eq) are performed similar to the above procedure.

After each coupling and deprotection the peptide resin is washed DMF (2×5 Vol.), DCM (2×5 Vol.) and DMF (2×5 Vol.) respectively.

Each coupling and deprotection is monitored by Bromophenol blue test.

After completion of synthesis, peptide resin is washed with Methanol (2×5 Vol.), MTBE (2×5 Vol.) and DCM (2×5 Vol.) respectively.

Resin cleavage: Peptide is cleaved from the resin using 1% TFA/DCM (3×10 Vol.), 5 minutes for each time. Cleavage fractions are collected neutralized with pyridine (1:1 Volume ratio to TFA in each cleavage). Combined cleavage fractions are concentrated under vacuo at 30-38° C. Resulting solution is co-evaporated with ethanol (2×5 Vol.) up to minimum volume and slurry is added to purified water, stirred for 18 h, solid is filtered, washed with purified water and suck dried for 24 h and then dried in VTD under reduced pressure at 45-50° C. to afford white solid. Yield: 123.12 g, 85.4%

IX. Synthesis of Boc-Asn(Trt)-Asp(OtBu)-Glu(OtBu)-Cys(Trt)-Glu(OtBu)-Leu-Cys(Acm)-Val-OH:—

### Example 14

2-CTC resin (100 g, loading capacity-0.9 mmol/g) is charged in a 3000 mL clean dry peptide synthesizer vessel. Resin is swelled with DCM (10V), 30 min, 2 times each.

1^{st} amino acid coupling: First amino acid Fmoc-Val-OH (91.63 g, 3.0 eq) with DIPEA (69.79 mL, 6.0 eq) in DCM (10 Vol.) is added to pre-swelled resin; agitated for 6 h. Drained the solvent, washed the resin with DCM (2×5 Vol.) Capping solution of DCM: MeOH: DIPEA (85%:10%:5%, 10 Vol.) is charged to above resin, agitated for 15 min for capping the resin. Drained the solvent and resin is washed with DCM (2×5 Vol.) and DMF (2×5 Vol.) respectively.

Fmoc deprotection: Deprotection of Fmoc group is performed by adding 20% piperidine in DMF (2×10 Vol.) for 10 min followed by 15 min respectively. Drained the solvent, washed resin with DMF (2×5 Vol.), DCM (2×5 Vol.) and DMF (2×5 Vol.) respectively.

2^{nd} amino acid coupling: The pre-activated Fmoc-Cys(Acm)-OH (74.60 g, 2.0 eq) with HOBt (24.32 g, 2.0 eq) and DIC (22.71 mL, 2.0 eq) in DMF (10V) is added to loaded resin, agitated for 3 h. Drained solvent and peptide resin is washed with DMF (2×5 Vol.), DCM (2×5 Vol.), DMF (2×5 Vol.) respectively.

Sequential coupling of next six amino acids Fmoc-Leu-OH (63.61 g, 2.0 eq), Fmoc-Glu(OtBu)-OH (76.58 g, 2.0 eq), Fmoc-Cys(Trt)-OH (105.42 g, 2.0 eq), Fmoc-Glu(OtBu)-OH (76.58 g, 2.0 eq), Fmoc-Asp(OtBu)-OH (74.06 g, 2.0 eq), Boc-Asn(Trt)-OH (80.54 g, 2.0 eq), are performed similar to the above procedure.

After each coupling and deprotection the peptide resin is washed DMF (2×5 Vol.), DCM (2×5 Vol.) and DMF (2×5 Vol.) respectively.

Each coupling and deprotection is monitored by Bromophenol blue test.

After completion of synthesis, peptide resin is washed with Methanol (2×5 Vol.), MTBE (2×5 Vol.) and DCM (2×5 Vol.) respectively.

Resin cleavage: Peptide is cleaved from the resin using 1% TFA/DCM (3×10 Vol.), 5 minutes for each time. Cleavage fractions are collected neutralized with pyridine (1:1 Volume ratio to TFA in each cleavage). Combined cleavage fractions are concentrated under vacuo at 30-38° C. Resulting solution is co-evaporated with ethanol (2×5 Vol.) up to minimum volume and slurry is added to purified water, stirred for 18 h, solid is filtered, washed with purified water and suck dried for 24 h and then dried in VTD under reduced pressure at 45-50° C. to afford white solid.

Yield: 135.62 g, 86.2%

X. Coupling of Fmoc-Asn(Trt)-Val-Ala-Cys(Trt)-Thr(tBu)-Gly-OH with Cys(Acm)-Leu-OtBu

### Example 15

To a stirred solution of Fmoc-Asn(Trt)-Val-Ala-Cys(Trt)-Thr(tBu)-Gly-OH (35.0 gm, 1.0 eq) in DMF (10 Vol.) is added Cys(Acm)-Leu(OtBu) (15.5 g, 1.2 eq) and HOAt (5.45 g, 1.2 eq) by maintaining temperature 5 to 10° C. EDC HCl (7.68 g, 1.5 eq) is added in a lot wise manner at same temperature. Resulting reaction mixture is stirred for about 3 to 4 hrs at 25-30° C. After completion of the reaction, reaction mixture is poured into pre-cooled 2.5% NaHCO₃ solution (1.4 L, 40 Vol.), stirred for a period of 15-30 minutes. Solid is filtered under vacuum, washed with purified water (350 mL). The obtained solid is leached with 1% KHSO₄ solution (350 mL, 10 Vol.) and ACN: Water (5:10, 525 mL, 15 V), filtered and dried under high vacuum to obtain off white solid. Yield: 41.52 g, 94.00%

XI. Deprotection to form Asn(Trt)-Val-Ala-Cys(Trt)-Thr(tBu)-Gly-Cys(Acm)-Leu-OtBu

### Example 16

To an ice cooled stirred solution of Fmoc-Asn(Trt)-Val-Ala-Cys(Trt)-Thr(tBu)-Gly-Cys(Acm)-Leu-OtBu (42 g, 1.0 eq) in DMF (420 mL, 10 Vol.), Tertiary butyl amine (3.86 mL, 1.5 eq) is added, stirred for 15 min at 5-10° C. n-heptane (210 mL, 5 Vol.) is charged into reaction mixture, stirred for 4 hr at 25-30° C. After completion of reaction, n-heptane layer is separated and DMF layer is cooled to 0-10° C., pH is adjusted to 3-4 by using 20% KHSO₄ solution (210 mL) and filtered. Filtrate is washed with 30% MTBE/Heptane (3×5 Vol.) and 10% Ethyl acetate/Heptane (3×5 Vol.). Aqueous layer is cooled to 0-10° C., pH is adjusted to 6-7 by using 2.5% NaHCO₃ solution (250 mL). Desired product is extracted using DCM (1×10 Vol., 2×5 Vol.). Combined organic layer is dried over anhydrous Na₂SO₄, filtered and concentrated under reduced vacuum, solid isolated in MTBE (10 Vol.) to afford off white solid.

Yield: 31.63 g, 87.10%

XII. Coupling of Boc-Asn(Trt)-Asp(OtBu)-Glu(OtBu)-Cys(Trt)-Glu(OtBu)-Leu-Cys(Acm)-Val-OH with Asn(Trt)-Val-Ala-Cys(Trt)-Thr(tBu)-Gly-Cys(Acm)-Leu-OtBu

### Example 17

To a stirred solution of Boc-Asn(Trt)-Asp(OtBu)-Glu(OtBu)-Cys(Trt)-Glu(OtBu)-Leu-Cys(Acm)-Val-OH (36.61 g, 1 eq) in DMF (25 vol), Asn(Trt)-Val-Ala-Cys(Trt)-Thr(tBu)-Gly-Cys(Acm)-Leu-OtBu (30.0 g, 1 eq), Cl-HOBt (4.25 g, 1.2 eq) and HBTU (9.51 g, 1.2 eq) are added by maintaining temperature −5 to 10° C. DIPEA (7.30 mL, 2.0 eq) is added slowly by maintaining pH 6-7. Stirring is continued for 30 minutes at −5 to 10° C. The reaction mixture is stirred for 3-4 hrs at 25-30° C. After completion of the reaction, reaction mixture is poured into precooled 0.5N HCl (100V) solution, stirred for a period of 15-30 minutes. Solid is filtered under vacuum, washed with 0.5N HCl (2×25 vol), purified water (2×25 vol), Sat. NaHCO₃ (2×25 vol), purified water (2×25 vol) and MTBE (2×25 vol). The solid is dried under high vacuum to obtain fully protected peptide as white solid (˜78% yield) with LCMS purity >95%.

C. Global Deprotection:

### Example 18

After completing said four compound couplings; to peptide resin a pre-cooled mixture of TFA: TIPS: Phenol: H₂O (88%:5.0%:5.0%:2.0%) (0-5° C.) is added. Resulting mixture is stirred for 4 h at 20-25° C. Reaction mixture is filtered to pre-cooled (10° C.) MTBE (40 vol.), solid is precipitated out. Solid is collected by vacuum filtration and washed with MTBE (2×20 vol.). Solid is dried under nitrogen atmosphere.

### Example 19

To a pre-cooled cleavage mixture-TFA: TIPS: Phenol: Dithiothreitol: Thioanisole: H₂O (82.5%:2.5%:2.5%:2.5%:5%:5%, 10 Vol.) is added linear protected peptide (40 g) at 5-10° C. Resulting mixture is stirred for 5 h at 20-25° C. After completion of the reaction, reaction mass is poured into pre-cooled (10° C.) MTBE (100 Vol.), stirred for a period of 15-30 minutes. Solid is filtered under vacuum, washed with MTBE (2×5 Vol.). The obtained solid is leached with DCM (10V×2), filtered and dried to afford off white solid. Yield: 23.2 g, 97.3%

XIII. Oxidation with Charcoal (1st Disulfide Bridge Formation)

### Example 20

23 g of peptide is taken in 0.2M ammonium acetate in water (23 L, 1000 Vol.), p^{H} of solution is adjusted with aq. ammonia solution ˜8-9 and is added 1% w/w charcoal granular (23 g). Reaction mixture is stirred for 16 h. After completion of reaction; reaction mixture is filtered and purified by using preparative HPLC using 20 mM Ammonium Acetate in water/acetonitrile to provide pure mono disulfide bridge peptide. Fractions with purity 97% are pooled and lyophilized to afford white solid.

Yield: 14 g, 61.13% (Purity >97%).

XII. Iodine Oxidation (2^{nd} Disulfide Bridge Formation):

### Example 21

The lyophilized monocyclized peptide (solid is dissolved in Aq. 5% AcOH, 0.5M Iodine solution (1.2 eq.) in methanol is added in a drop wise manner with vigorous stirring for 1 h at 20° C. to about 35° C. Upon completion of reaction, the excess of iodine is quenched with Diaion PAF 308 F resin. Reaction mixture is filtered and directly is subjected for reverse phase prep. HPLC purification using octadecylsilane as stationary phase with 0.3%-0.5% acetic acid aqueous solution/acetonitrile as a mobile phase. Pure fractions are pooled and lyophilized to afford white Plecanatide (purity>98%).

### Example 22

The lyophilized monocyclized peptide (10 g) is dissolved in 1% AcOH in water (10 L) and is added Iodine (1.7 g, 1.2 eq) as a 5% solution in acetonitrile. Reaction mixture is stirred for 1.5 hr. Upon completion of reaction, excess of iodine is quenched with Diaion PAF 308 F resin (40 g). Reaction mixture is filtered and subjected for reverse phase prep. HPLC purification using Octadecylsilane as stationary phase is carried out using following RP-HPLC conditions,

### Condition 1) HCOOH Buffer

Solvent A—1% HCOOH in Water pH 6.5 using Ammonia solution
Solvent B:—Acetonitrile:Water (1:1)
Gradient—

| Time (min) | 2 | 15 | 15 | 5 | 15 |
|---|---|---|---|---|---|
| Solvent B % | 0 | 0 to 20 | 20 | 20 to 23 | 23 |

Condition 2) CH₃COOH Buffer
Solvent A:—1% of CH₃COOH in Water pH 6.5 using Ammonia solution
Solvent B:—Acetonitrile:Water (1:1)
Gradient:

| Time (min) | 2 | 15 | 15 | 5 | 15 |
|---|---|---|---|---|---|
| Solvent B % | 0 | 0 to 20 | 20 | 20 to 23 | 23 |

Pure fractions (>98% HPLC purity) are pooled together and lyophilized and subjected for desalting using following RP-HPLC condition, Condition 3)
Solvent A:—0.05% CH₃COOH in water
Solvent B:—Water:IPA (3:2)

| Time (min) | 2 | 35 | 10 |
|---|---|---|---|
| Solvent B % | 0 | 0 to 75 | 100 |

Yield: 4.5 g, 48.91% (HPLC purity>98%).

Thus the present invention provides a facile and economical method of synthesis Plecanatide in high yield and with less impurities; adoptable on an industrial scale.

1. A method of synthesis of Plecanatide, said method comprising acts of,
a) preparing peptide compound of formula A and deprotecting to obtain peptide compound of formula A1,
Formula A:
Fmoc-Cys(Acm)-Leu(OtBu)
Formula A1:
Cys(Acm)-Leu(OtBu)
b) coupling peptide compound of formula A1 with peptide compound of formula B or formula D and deprotecting to obtain peptide compound of formula B1 and D1,
Formula B:
Fmoc-Ala-Cys(Trt)-Thr(tBu)-Gly-OH
Formula B1:
Ala-Cys(Trt)-Thr(tBu)-Gly-Cys(Acm)-Leu(OtBu)
Formula D:
Fmoc-Asn(Trt)-Val-Ala-Cys(Trt)-Thr(tBu)-Gly-OH
Formula D1:
Asn(Trt)-Val-Ala-Cys(Trt)-Thr(tBu)-Gly-Cys(Acm)-
Leu(OtBU)
c) coupling the peptide compound of formula B1 or formula D1 with peptides of formula C or formula E and deprotecting to obtain compound of formula F,
Formula C:
Boc-Asn(Trt)-Asp(OtBu)-Glu(OtBu)-Cys(Trt)-Glu
(OtBu)-Leu-Cys(Acm)-Val-Asn(Trt)-Val-OH
Formula E:
Boc-Asn(Trt)-Asp(OtBu)-Glu(OtBu)-Cys(Trt)-Glu
(OtBu)-Leu-Cys(Acm)-Val-OH
Formula F:
Boc-Asn(Trt)-Asp(otBu)Glu(OtBu)-Cys(Trt)-Glu
(otBu)-Leu-Cys(Acm)-Val-Asn(Trt)-Val-Ala-Cys
(Trt)-Thr(tBu)-Gly-Cys(Acm)-Leu-OtBu
d) oxidising the deprotected compound of formula F with charcoal to form disulphide bridge between two Cysteine amino acids at 4^{th} and 12^{th} positions to obtain compound of formula G and lyophilizing; 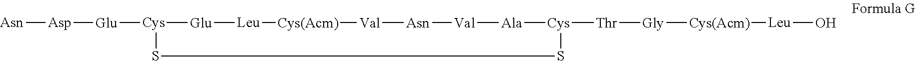
e) treating the lyophilized compound of formula G with Iodine to form second disulphide bridge between two cysteine aminoacids at 7^{th} and 15^{th} positions and quenching with Diaion PAF 308 F resin and purifying to obtain Plecanatide.

2. The method of synthesis of Plecanatide as claimed in claim 1, wherein the peptide compound of formula A1: Cys(Acm)-Leu(OtBu) is treated with peptide compound of formula B: Fmoc-Ala-Cys(Trt)-Thr(tBu)-Gly-OH and deprotecting to obtain peptide compound of formula B1:Ala-Cys(Trt)-Thr(tBu)-Gly-Cys(Acm)-Leu(OtBu).

3. The method of synthesis of Plecanatide as claimed in claim 1, wherein the peptide compound of formula A1: Cys(Acm)-Leu(OtBu) is treated with peptide compound of formula D: Fmoc-Asn(Trt)-Val-Ala-Cys(Trt)-Thr(tBu)-Gly-OH and deprotecting to obtain peptide compound of formula D1: Asn(Trt)-Val-Ala-Cys(Trt)-Thr(tBu)-Gly-Cys(Acm)-Leu(OtBu).

4. The method of synthesis of Plecanatide as claimed in claim 1, wherein the peptide compound of formula B1: Ala-Cys(Trt)-Thr(tBu)-Gly-Cys(Acm)-Leu(OtBu) is treated with peptide compound of formula C: Boc-Asn(Trt)-Asp(OtBu)-Glu(OtBu)-Cys(Trt)-Glu(OtBu)-Leu-Cys(Acm)-Val-Asn(Trt)-Val-OH to obtain peptide compound of formula K: Boc-Asn(Trt)-Asp(otBu)Glu(OtBu)-Cys(Trt)-Glu(otBu)-Leu-Cys(Acm)-Val-Asn(Trt)-Val-Ala-Cys(Trt)-Thr(tBu)-Gly-Cys(Acm)-Leu-OtBu.

5. The method of synthesis of Plecanatide as claimed in claim 1, wherein the peptide compound of formula D1: Asn(Trt)-Val-Ala-Cys(Trt)-Thr(tBu)-Gly-Cys(Acm)-Leu(OtBu) is treated with peptide compound of formula E: Boc-Asn(Trt)-Asp(OtBu)-Glu(OtBu)-Cys(Trt)-Glu(OtBu)-Leu-Cys(Acm)-Val-OH to obtain peptide compound of formula K: Boc-Asn(Trt)-Asp(otBu)Glu(OtBu)-Cys(Trt)-Glu(otBu)-Leu-Cys(Acm)-Val-Asn(Trt)-Val-Ala-Cys(Trt)-Thr(tBu)-Gly-Cys(Acm)-Leu-OtBu.

6. The method of synthesis of Plecanatide as claimed in claim 1, wherein the coupling of peptide compound of formula A1 with peptide compound of formula B or formula D is with HOAt in presence of EDC·HCl.

7. The method of synthesis of Plecanatide as claimed in claim 1, wherein the charcoal is 1% w/w.

8. The method of synthesis of Plecanatide as claimed in claim 1, wherein the purification is by reverse-phase high-pressure liquid chromatography.

9. The method of synthesis of Plecanatide as claimed in claim 8, wherein the purification by reverse-phase high-pressure liquid chromatography is carried out under conditions selected from a group comprising—
(a) Solvent A—1% HCOOH in Water with pH 6.5 and Solvent B—Mixture of Acetonitrile and Water in ratio 1:1,
(b) Solvent A—1% of CH₃COOH in Water with pH 6.5 and Solvent B—Mixture of Acetonitrile and Water in ratio 1:1, and
(c) Solvent A—0.05% CH₃COOH in water and Solvent B—Mixture of Water and IPA in ratio 3:2.

10. A method of preparation of Plecanatide comprising acts of
a) coupling peptide compounds selected from a group containing
(i) Formula A1:
Cys(Acm)-Leu(OtBu),
(ii) Formula B:
Fmoc-Ala-Cys(Trt)-Thr(tBu)-Gly-OH,
(iii) Formula B1:
Ala-Cys(Trt)-Thr(tBu)-Gly-Cys(Acm)-Leu(OtBu),
(iv) Formula D:
Fmoc-Asn(Trt)-Val-Ala-Cys(Trt)-Thr(tBu)-Gly-OH,
(v) Formula D1:
Asn(Trt)-Val-Ala-Cys(Trt)-Thr(tBu)-Gly-Cys(Acm)-
Leu(OtBU),
(vi) Formula C:
Boc-Asn(Trt)-Asp(OtBu)-Glu(OtBu)-Cys(Trt)-Glu
(OtBu)-Leu-Cys(Acm)-Val-Asn(Trt)-Val-OH,
and
(vii) Formula E:
Boc-Asn(Trt)-Asp(OtBu)-Glu(OtBu)-Cys(Trt)-Glu
(OtBu)-Leu-Cys(Acm)-Val-OH;
to form compound of formula F: Boc-Asn(Trt)-Asp(otBu)Glu(OtBu)-Cys(Trt)-Glu(otBu)-Leu-Cys(Acm)-Val-Asn(Trt)-Val-Ala-Cys(Trt)-Thr(tBu)-Gly-Cys(Acm)-Leu-OtBu,
b) oxidising the deprotected compound of formula F with charcoal to form disulphide bridge between two Cysteine amino acids at 4^{th} and 12^{th} positions to obtain compound of formula G and lyophilizing, and 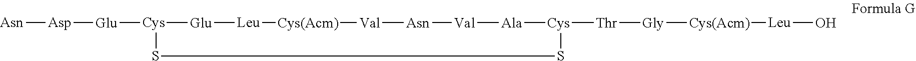
c) treating the lyophilized compound of formula G with Iodine to form second disulphide bridge between two cysteine aminoacids at 7^{th} and 15^{th} positions and quenching with Diaion PAF 308 F resin and purifying to obtain Plecanatide.

11. The method of synthesis of Plecanatide as claimed in claim 1, wherein the yield of Plecanatide is ranging from about 60-70% and purity is ranging from 96%-98.5%.

12. (canceled)

13. (canceled)

14. (canceled)

15. (canceled)

16. The method of synthesis of Plecanatide as claimed in claim 2, wherein the peptide compound of formula B1: Ala-Cys(Trt)-Thr(tBu)-Gly-Cys(Acm)-Leu(OtBu) is treated with peptide compound of formula C: Boc-Asn(Trt)-Asp(OtBu)-Glu(OtBu)-Cys(Trt)-Glu(OtBu)-Leu-Cys(Acm)-Val-Asn(Trt)-Val-OH to obtain peptide compound of formula K:Boc-Asn(Trt)-Asp(otBu)Glu(OtBu)-Cys(Trt)-Glu(otBu)-Leu-Cys(Acm)-Val-Asn(Trt)-Val-Ala-Cys(Trt)-Thr(tBu)-Gly-Cys(Acm)-Leu-OtBu.

17. The method of synthesis of Plecanatide as claimed in claim 3, wherein the peptide compound of formula D1: Asn(Trt)-Val-Ala-Cys(Trt)-Thr(tBu)-Gly-Cys(Acm)-Leu(OtBu) is treated with peptide compound of formula E: Boc-Asn(Trt)-Asp(OtBu)-Glu(OtBu)-Cys(Trt)-Glu(OtBu)-Leu-Cys(Acm)-Val-OH to obtain peptide compound of formula K: Boc-Asn(Trt)-Asp(otBu)Glu(OtBu)-Cys(Trt)-Glu(otBu)-Leu-Cys(Acm)-Val-Asn(Trt)-Val-Ala-Cys(Trt)-Thr(tBu)-Gly-Cys(Acm)-Leu-OtBu.

18. The method of synthesis of Plecanatide as claimed in claim 10, wherein the yield of Plecanatide is ranging from about 60-70% and purity is ranging from 96%-98.5%.

19. A peptide compound for preparation of Plecanatide selected from the group consisting of:
Formula B:
F moc-Ala-Cys(Trt)-Thr(tBu)-Gly-OH;
Formula C:
Boc-Asn(Trt)-Asp(OtBu)-Glu(OtBu)-Cys(Trt)-Glu
(OtBu)-Leu-Cys(Acm)-Val-Asn(Trt)-Val-OH;
Formula D:
F moc-Asn(Trt)-Val-Ala-Cys(Trt)-Thr(tBu)-Gly-OH;
and
Formula E:
Boc-Asn(Trt)-Asp(OtBu)-Glu(OtBu)-Cys(Trt)-Glu
(OtBu)-Leu-Cys(Acm)-Val-OH.

20. The peptide compound as claimed in claim 19, wherein the peptide compound is Formula B: F moc-Ala-Cys(Trt)-Thr(tBu)-Gly-OH.

21. The peptide compound as claimed in claim 19, wherein the peptide compound is Formula C: Boc-Asn(Trt)-Asp(OtBu)-Glu(OtBu)-Cys(Trt)-Glu(OtBu)-Leu-Cys(Acm)-Val-Asn(Trt)-Val-OH.

22. The peptide compound as claimed in claim 19, wherein the peptide compound is Formula D: F moc-Asn(Trt)-Val-Ala-Cys(Trt)-Thr(tBu)-Gly-OH.

23. The peptide compound as claimed in claim 19, wherein the peptide compound is Formula E: Boc-Asn(Trt)-Asp(OtBu)-Glu(OtBu)-Cys(Trt)-Glu(OtBu)-Leu-Cys(Acm)-Val-OH.
